(19) United States
(12) Patent Application Publication
(10) Pub. No.: US 2008/0249467 A1
(43) Pub. Date: Oct. 9, 2008

(54) Device and Method for Safe Access to a Body Cavity

(76) Inventors: Daniel Rogers Burnett, San Francisco, CA (US); Gregory Hall, Redwood City, CA (US)

Correspondence Address: SHAY GLENN LLP, 2755 CAMPUS DRIVE, SUITE 210, SAN MATEO, CA, 94403, US

(21) Appl. No.: 12/098,355

(22) Filed: Apr. 4, 2008

Related U.S. Application Data
(60) Provisional application No. 60/921,974, filed on Apr. 5, 2007.
(60) Provisional application No. 60/926,749, filed on Apr. 30, 2007.

(51) Int. Cl. A61M 5/46

(52) U.S. Cl. 604/117

(57) ABSTRACT

An anatomical space access device having an elongate body; an insertion tip at a distal end of the elongate body; an anatomical space sensor disposed at the distal end of the elongate body, the sensor being adapted to sense a parameter identifying an anatomical space other than a vasculature space and to generate a signal; and an indicator operatively connected to the sensor to receive the signal and to indicate access of the sensor to the anatomical space. The invention also provides a method for providing access to an anatomical space outside of a vasculature space, including the following steps: inserting a distal end of an instrument through a tissue volume into the anatomical space outside of a vasculature space, the instrument comprising an anatomical space sensor; and generating a location indication of the anatomical space sensor.

### CROSS-REFERENCE TO RELATED APPLICATIONS

This application claims the benefit of U.S. Provisional Patent Applications No. 60/921,974, filed Apr. 5, 2007 to Burnett, entitled “Safety Access Device, Fluid Output Monitor & Peritoneal Organ Preservation”; and No. 60/926,749, filed Apr. 30, 2007 to Burnett, entitled “Device and Method for Safe Abdominal Access,” the disclosures of which are incorporated by reference herein in their entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

In a non-image-guided peritoneal access procedure, the interventionalist is left to assume, or hope, that they have accessed the correct cavity prior to the desired intervention and have done so in a manner that has not harmed adjacent structures. The same guesswork applies to interventional access to other body cavities. Little work has been done, though, in improving the safety of the access and in reporting that correct access has been obtained during invasive procedures.

In the current state-of-the-art, laparoscopic trocar placement, whether visualized or not, has been fraught with complications. The main reason for this is several-fold including: (1) lack of appropriate tenting of the abdominal cavity, (2) lack of feedback with respect to the driving force behind insertion and (3) use of a sharp or damaging cutting tip.

Prior systems for determining the depth, position or location of insertable medical instruments fail to provide sufficient information about the tissue, cavity or other location of the instrument within the body. For example, U.S. Pat. No. 5,425,367 describes a catheter depth, position and orientation location system using two orthogonally disposed sets of coils. Instrument depth of insertion is determined by sensing signal strength from the coils. U.S. Pat. No. 5,709,661 describes a system with a catheter displacement system that monitors advancement and rotation of the catheter using an optical encoder or magnets. U.S. Pat. No. 6,019,729 describes a catheter with a pressure sensor on its distal end to detect obstacles during advancement. U.S. Pat. No. 6,551,302 describes a catheter system with surface contact detection using pressurized saline. In U.S. Pat. No. 6,304,776, oscillating voltage is used to detect contact between a catheter tip and tissue. U.S. Pat. No. 6,704,590 uses a Doppler sensor on an arterial catheter to sense blood flow turbulence to identify catheter location. Finally, U.S. Pat. No. 6,807,444 describes a system using tissue impedance to differentiate between a tumor and normal tissue.

While tenting is called for during Veress needle insertion and trocar insertion, it is typically performed by manually grasping the abdominal cavity distant from the site of puncture so that the tissues to be entered are still able to be driven deeper and closer to the at-risk organs. Furthermore, there is no good indicator as to the appropriate force required for abdominal entry which is particularly true of the blunt trocars (require more force and rotating motion).

### SUMMARY OF THE INVENTION

In reviewing the obstacles of providing safe access to the peritoneal cavity, it becomes clear that over- and under-insertion of invasive instrumentation is a major issue. During catheter placement in the peritoneal cavity, for example, even with manual tenting of the abdomen and an optically-guided trocar, damage to major abdominal vessels, e.g., the aorta or iliac arteries, and bowel has been reported. One aspect of the invention is a method and device for safe access of the peritoneal cavity. The improved safety of the current invention is based, in part, on the ability of the access system to detect and report entry into the peritoneal cavity. Additional safety may be provided by the ability to tent the abdomen in a focused manner directly at the site of trocar insertion, by the use of a blunt-tipped trocar and/or by the use of a force-gauge or force-limiter to help guide the level of insertion force (which is frequently excessive or inadequate). In one embodiment, additional sensing capabilities may be incorporated as well to optimize the desired intervention or therapy to be delivered.

The tenting mechanism of the current invention may involve capturing the tissue around the site of insertion (via superficial puncture, suction, use of adhesives, etc.) at one or more sites and then applying an upward force during cavity entry. This tissue capture, in one embodiment, is fully, or nearly fully, circumferential to the access site to provide optimal tenting directly adjacent to the site of puncture. In addition, the tissue capture mechanism may, after application to the abdominal skin, allow for single-handed application of abducting force while the trocar or entry device is driven into the cavity. The tenting device may also consist of multiple components such that the grasping component may be detached for the low-profile tissue capture element such that remains at the site of cavity entry. The tenting may also be reversible and allow for immediate removal of the entire device once access has been obtained. In this embodiment, the tissue puncture may be released, the suction may be deactivated, the adhesive may be dissolved, etc., once the trocar has been inserted into the cavity and the at-risk organs have been spared.

A force gauge or force limiting mechanism may also be employed along with the above-mentioned feature or on its own in safely accessing the peritoneal, or any other, cavity. This component provides feedback to the user and prevents application of excessive force during cavity entrance. In one embodiment, the device alerts the user to both inadequate and excessive pressures via tactile, visual, auditory, or other stimulus. The device may also be capable of alerting the user to slightly inadequate or slightly excessive forces application. In the peritoneal embodiment, for example, the blunt-tipped trocar may be driven by a handle or other component which signals the amplitude of the force along the axis of the insertion device. This signal may be as simple as a circuit which is closed with appropriate pressure and not with excessive or inadequate pressure. In another embodiment utilizing the asymmetric peritoneal insertion device, the feedback to the user may be based on rotation. With asymmetric blunt trocars, safe insertion requires forward motion while rotating the trocar itself. In one example of this embodiment, the spring-loaded or shape-memory component of the handle will allow the interdigitating elements of the trocar to engage and permit application of rotational forces only when the appropriate force is applied. Too much force will overshoot the appropriate engagement site and inadequate force will undershoot the engagement site.

This force gauge feature, however, could utilize any mechanism to report the appropriate force range and to prevent over-insertion of the penetrating element. The feature could also be used in the accessing of other body cavities, e.g., bone marrow biopsies, lumbar punctures, orthopedic screwing/plating or other manipulations of bone, thoracentesis, paracentesis, etc.

Some embodiments may include a force gauge or force-limiter that utilizes the tenting handle to engage or disengage the rotational forces, as in threaded and/or asymmetric blunt trocar. In this embodiment the penetrating element may only advance when the appropriate force is applied in abducting the tenting handle. This safety feature will help ensure that the appropriate tenting force is applied while the penetrating element is advanced.

One aspect of the invention provides an anatomical space access device having an elongate body; an insertion tip at a distal end of the elongate body; an anatomical space sensor disposed at the distal end of the elongate body, the sensor being adapted to sense a parameter identifying an anatomical space other than a vasculature space and to generate a signal; and an indicator operatively connected to the sensor to receive the signal and to indicate access of the sensor to the anatomical space. The insertion tip may be blunt or sharp. The sensor may include, e.g., an electrical property sensor; a tissue compliance sensor; a pressure sensor; and/or an insertion force sensor.

In some embodiments, the elongate body includes a sheath, with the device further including a trocar disposed within the sheath. The sheath may have a weighted tip. In other embodiments, the elongate body includes a trocar, with the device further including a sheath surrounding the trocar. The trocar may be threaded.

Some embodiments of the device include a tenting mechanism having a tissue attachment mechanism and a handle. The elongate body may be disposed within the tenting mechanism and may possibly be movably engaged with the tenting mechanism, such as by the interaction of threads on the elongate body and on the tenting mechanism.

Some embodiments of the device include a tissue incision tool disposed within the tenting mechanism, such as a spring biasing a sharp edge of the tool. In this embodiment, the tissue incision tool is ideally utilized to control the superficial skin incision and then removed from the lumen of the tenting mechanism to allow insertion of the penetrating element.

In some embodiments, the device includes a rotation force sensor. Other embodiments of the device include a rotation actuator engageable with the elongate body to rotate the elongate body and disengageable with the elongate body if a distally directed insertion force is below or above a threshold level. Alternatively, the rotation actuator may be disengaged automatically once cavity entry has been detected.

In some embodiments, the anatomical space sensor has a distal tip mechanically connected to a first proximal contact such that the distal tip and first proximal contact are movable with respect to the elongate body, the anatomical space sensor further including a second proximal contact connected to a proximal end of the elongate body, the first and second proximal contacts having an open position in which the contacts are not in contact and a closed position in which the contacts are in contact. The device in some embodiments may have an automated actuator operably connected to the elongate body to advance the elongate body only when the first and second proximal contacts are not in contact. The anatomical space sensor may also have a spring biasing the first and second proximal contacts toward the closed position.

Another aspect of the invention provides a method for providing access to an anatomical space outside of a vasculature space, such as a peritoneal cavity. The method includes the steps of inserting a distal end of an instrument through a tissue volume into the anatomical space outside of a vasculature space, the instrument comprising an anatomical space sensor; and generating a location indication of the anatomical space sensor.

Some embodiments of the method include the step of creating an opening in the tissue volume with the instrument. In embodiments of the method in which the instrument has a blunt tip, the step of creating an opening may include the step of advancing the blunt tip through the tissue volume.

In some embodiments, the step of generating a location indication includes the step of sensing a parameter with the anatomical space sensor, such as an electrical property, temperature, change in tissue compliance, and/or a breathing pressure waveform. Some embodiments of the method include the step of sensing an insertion force during the inserting step and optionally indicating insertion force information.

In some embodiments, the instrument further has a blunt tip, and the method includes the step pushing an anatomical structure (such as a bowel or vasculature) aside during the inserting step.

In embodiments in which the instrument includes an insertion trocar and a sheath, the method may also include the step of removing the trocar and sensor after the inserting and generating steps. In embodiments in which the instrument includes a sheath, the method may also include the step of removing the sheath and sensor after the inserting and generating steps.

Some embodiments include the step of tenting the tissue volume prior to the inserting step, such as by attaching a handle to the tissue volume and, optionally, inserting the instrument through the handle. Some embodiments add the step of preventing insertion of the instrument in the absence of a threshold tenting force.

In some embodiments, the inserting step includes the step of rotating the instrument and advancing the instrument distally, such as by rotating a handle with a handle rotation force, with the method further including disengaging transmission of the handle rotation force from the instrument if an advancement force is below a threshold level.

In some embodiments, the inserting step includes the step of inserting the instrument with an automated actuator, and the method further includes the step of automatically ceasing operation of the automated actuator when the distal end of the instrument reaches a target cavity.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which.

FIGS. 1A-D show an embodiment of an anatomical space access device in which an anatomical space sensor is incorporated into the access device body.

FIGS. 2A-E show another embodiment of the invention in which an anatomical space sensor is incorporated into a removable insertion trocar.

FIGS. 3A-E show an embodiment of the invention in which an anatomical space sensor 34 is incorporated into a removable elongate sheath.

FIGS. 4A-C show an external reader attached to an anatomical space access device.

FIGS. 5A-D show an embodiment of an anatomical space access device in which a continuous reader is incorporated into the access device.

FIGS. 6A-D shows an embodiment in which an intermittent reader is incorporated into the access device.

FIGS. 7A-E show an embodiment of the invention in which an anatomical space sensor is incorporated into a catheter.

FIGS. 8A-D show a tenting mechanism for use with the anatomical space access device of this invention.

FIGS. 9A-D show another embodiment of a tenting mechanism for use with the anatomical space access device of this invention.

FIGS. 10A-D show an insertion force sensor for use with an anatomical space access device of this invention.

FIGS. 11A-D show another embodiment of an insertion force sensor for use with an anatomical space access device, such as a catheter.

FIGS. 12A-D show a rotational feedback insertion sensor being used with an optional tenting mechanism and an anatomical space access device according to this invention.

FIGS. 13A-D show an embodiment of a tenting mechanism with a removable incision element for use with the anatomical space access devices of this invention.

FIGS. 14A-C show yet another embodiment of an anatomical space access device.

FIGS. 15A-C show an automated entry system in which an automated actuator turns a threaded trocar.

### DETAILED DESCRIPTION OF THE INVENTION

In one embodiment, the access system involves the use of a puncturing instrument in conjunction with a sensor at, or near, the distal tip of the instrument. This sensor may be capable of detecting changes within its environment in order to report that it has passed through the subcutaneous or muscular tissues surrounding the desired cavity, space or tissue and into the desired cavity, space or tissue itself. For example, one embodiment of the invention is a peritoneal access catheter which is capable of detecting differences between the vascular, extraperitoneal, intestinal and intraperitoneal spaces. This sensor may detect (1) changes in the physical properties surrounding the instrument such as pressure, acceleration, forces or other physical properties; (2) chemical changes surrounding the instrument, e.g., the presence or absence of compounds such as albumin, hemoglobin, glucose or the pH or other chemical properties; (3) changes in electrical properties such as conductance, resistance, impedance, capacitance, etc., of the tissues; (4) changes in the acoustic or vibratory properties of the tissues; (5) changes in optical properties such as refraction of light within the tissue; (6) changes in mechanical properties, such as pressure, shear forces or tissue compliance; and/or (7) changes in any other parameter that is able to be sensed via a sensor placed on, in, within or otherwise attached to or in communication with said instrument.

In any of the embodiments, the sensing element of the access device may be incorporated in the insertable instrument itself, may be introduced along with the instrument or may be external to the instrument and communicate with the tissue and/or cavity through a channel in said instrument. In one embodiment, the sensor is incorporated into either the instrument or its introducer and is able to provide immediate, definitive feedback that the correct body cavity has been accessed. For example, the electrical properties of blood are different from that of air, the epidermis, the subcutaneous space, the fascia and the adventia of the vessel. Thus, in accessing the femoral artery, one can slowly insert the arterial access device (e.g., a catheter with a sharp insertion trocar/needle) which incorporates a sensor in the catheter or insertion trocar/needle (in this case electrical) which will immediately report a change in the sensed parameter (in this case inductance, resistance, capacitance, etc.) indicating that the vessel has been entered. This same reading can then them be monitored continuously as the instrument is manipulated (e.g., the catheter is slid over the trocar/needle into the vessel) to ensure that the instrument does not migrate during manipulation and remains within the desired space.

Another embodiment uses heat differentials to guide a catheter/needle to the appropriate space/tissue. For example, by placing a cold pack on the skin over the femoral artery, a temperature differential will exist with the warmest location being in the intravascular space. A temperature sensing catheter can be guided to the warmest location which would be inside the vessel.

This sensing technique may be employed with virtually any invasive instrument to ensure correct placement via detection of changes in any of the aforementioned parameters (e.g., physical, chemical, thermal, electrical, acoustic/vibratory, optical or other parameter capable of being sensed) with the only requirement being that the target tissue or space within the body must have a sufficiently distinct sensor reading that it may be distinguished from its surrounding tissues. These invasive instruments may include, but are not limited to instruments, catheters or devices intended to access the following spaces/tissue: peritoneal cavity or fluid (e.g., paracentesis or peritoneal lavage); vascular fluid or space (arterial catheter, intravenous catheter, etc.); cerebrospinal fluid or space; pleural or pulmonary fluid or space (e.g., chest tubes); pericardial or cardiac space or tissue, urologic fluid or space (e.g., suprapubic catheters); gynecologic access (e.g., fallopian tubes or ovaries); gastrointestinal fluid or space (e.g., nasogastrostomy or gastrostomy tubes); ocular or bulbar tissues or spaces; neurological tissue or space (e.g., brain biopsy instruments); pathological tissue or space (e.g., abscess, hematoma, cyst, pseudocyst); bone marrow tissue or space; or any other tissues or spaces that may be accessed minimally invasively, percutaneously or through a natural orifice.

The sensing element may be disposable or reusable. The sensing element may be incorporated reversibly or irreversibly into the instrument itself, into the instrument's sheath, into the instrument's trocar, or kept external to said instrument with movement of gases, fluids or solids down the length of the instrument to the externally located sensor continuously or upon activation. The sensor may also communicate wirelessly from the instrument to an external receiver removing the requirement for a tethering cord and allowing for a disposable and reusable component. The controller/reader may alert the user that access has been obtained through tactile, auditory, visual or any other stimuli. The sensing may occur continuously or only upon command by the user (e.g., once they suspect that they are in the tissue or cavity).

The invention may be used with any instrument (such as a catheter, endoscope or trocar) that demands precise access to tissues, body cavities or spaces and/or requires automated, sensor-based intervention or therapy. Cavities to be accessed using this invention may include peritoneal, pleural, cerebrospinal, biliary, gastrointestinal, gastric, intestinal, urinary cavities, or pathologic tissues, and the anatomical space sensor may directly or indirectly detect entry into this cavity. The anatomical space to be accessed using this invention may include the cardiovascular, venous, arterial, lymphatic, ureteral cerebrospinal ventricular spaces, or pathologic spaces, and the anatomical space sensor may directly or indirectly detect entry into this space. Tissues to be accessed using this invention may include lung, liver, heart, bladder, brain, intestinal, pancreatic, splenic, vascular tissues, or pathologic spaces, and the anatomical space sensor may directly or indirectly detect entry into these tissues.

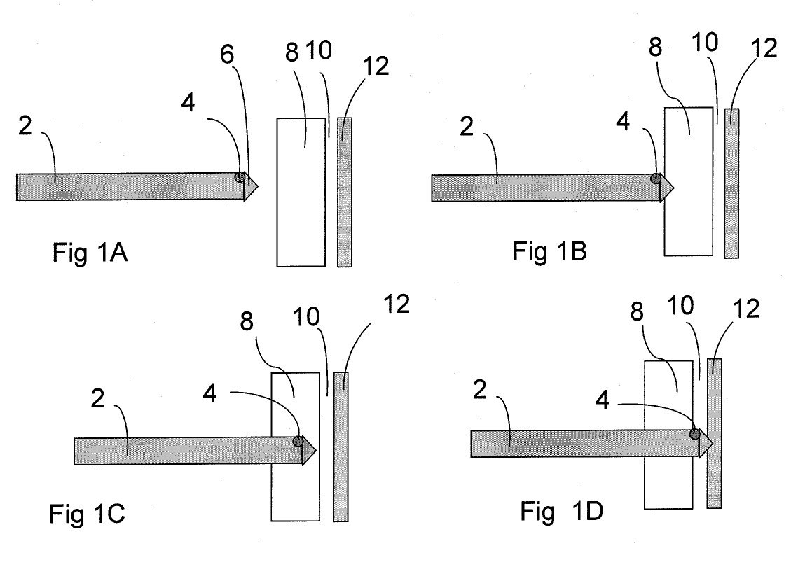

FIGS. 1A-D show an embodiment of an anatomical space access device in which an anatomical space sensor is incorporated into the access device body. In this embodiment, the instrument has an elongate body 2 (such as a needle or a trocar) with an anatomical space sensor 4 at its distal end which may intermittently or continuously provide information to the user to sense a parameter identifying an anatomical space. In this illustration, the distal tip 6 of the instrument is shown passing through the subcutaneous tissues and muscular layers 8 and entering a cavity 10 without harming or substantially penetrating the tissues 12 beneath the cavity 10. Examples where this illustration apply include: peritoneal cavity access, pleural cavity access, cerebrospinal cavity access, etc. In each of these cases, entrance into the space may be required, but the sensitive underlying tissues (the intestines/liver, lungs and spinal cord/brain, respectively) require a sensing technology to prevent over-insertion. In addition to information from the anatomical space sensor 4 (such as detection of (1) changes in the physical properties surrounding the instrument such as pressure, acceleration, forces or other physical properties; (2) chemical changes surrounding the instrument, e.g., the presence or absence of compounds such as albumin, hemoglobin, glucose or the pH or other chemical properties; (3) changes in the electrical properties such as conductance, resistance, impedance, capacitance, etc., of the tissues; (4) changes in the acoustic or vibratory properties of the tissues; (5) changes in optical properties such as refraction of light within the tissue to detect instrument entrance into the cavity; and/or (6) changes in the thermal properties of the tissues), the same sensor, or another sensor, may be capable of detecting other components that signal an issue may have occurred during entry. For example, when used with a peritoneal catheter the sensor, or another sensor, may be able to detect the presence of fecal matter or blood which would indicate that even though the cavity may have been entered, the catheter may been over-inserted or is not in its correct position. This positive feedback related to instrument entry and negative feedback with respect to possible incorrect positioning of the instrument, in combination, provide confidence to the user not only that the correct cavity, space or tissues have been accessed but that no complications have arisen during the access procedure.

One example of this embodiment is a peritoneal access catheter with an electrical inductance sensor at its tip. The subcutaneous space has a different inductance compared to the peritoneal space which also has a different inductance than the intestinal lumen. In accessing the peritoneal cavity, the catheter may be advanced until the subcutaneous tissue inductance readings change to the peritoneal cavity inductance levels. Once the peritoneal cavity is sensed, based on the change in electrical properties, the catheter then provides feedback that the cavity has been accessed. In the event that the catheter is over-inserted into the bowel, the inductance will be sufficiently lower than that found in the subcutaneous tissue or peritoneal space and this complication can be rapidly reported. In addition, iron-rich blood has a higher inductance than any of the other tissues and exposure to concentrated blood can be quickly reported if the catheter experiences this fluid. The cutoff may be set so that dilute blood does not trigger the sensor since minor capillaries may be ruptured in the normal access procedure. This same technique may be used, in reverse, to purposefully access the vascular space. In fact, most tissues have characteristic electrical properties and virtually any tissue, cavity or space may be accessed through monitoring for this signal during instrument insertion. The access device may be used to access any body tissue, space, or cavity and may do so with feedback from any of the sensors detailed above or any other sensing technology.

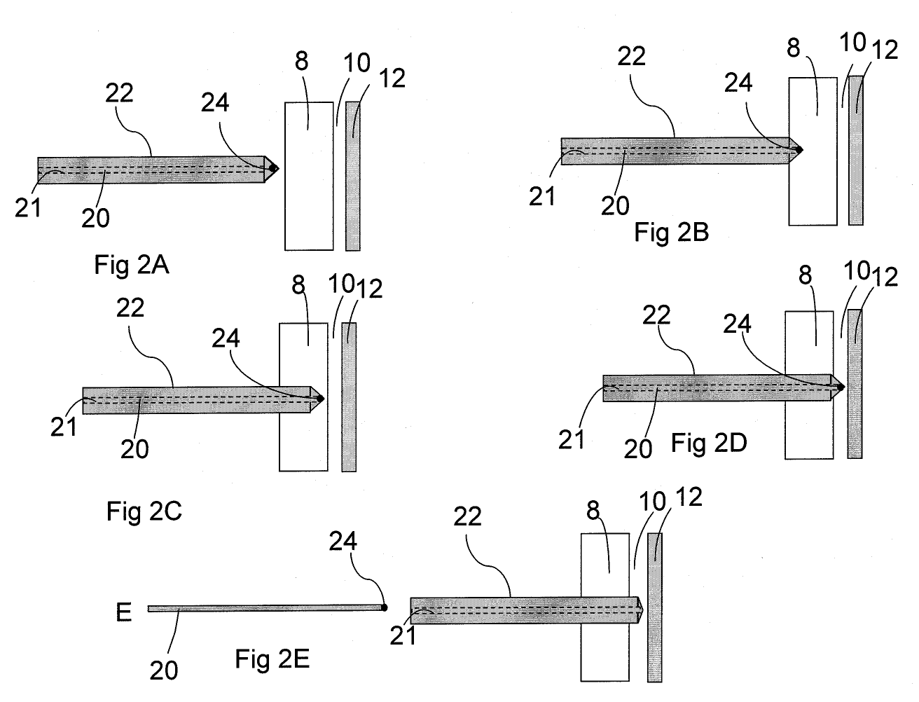

FIGS. 2A-E show another embodiment of the invention. In this embodiment, the anatomical space sensor 24 is disposed at the distal end of a removable insertion trocar 20 disposed in a channel 21 within a catheter 22 or other elongate body. Once the tissue, space, or cavity has been accessed, the insertion trocar 20 may be removed and the catheter 22 advanced or left in place to allow access to the tissue or space 10 for the intervention.

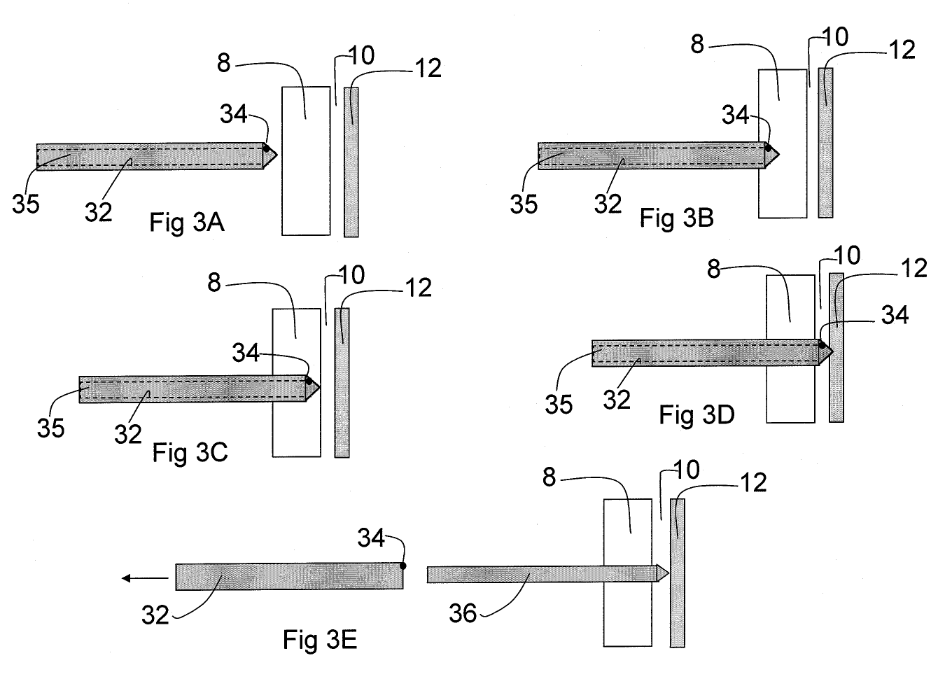

FIGS. 3A-E show an embodiment of the invention in which an anatomical space sensor 34 is incorporated into a removable elongate sheath 32. In this embodiment, the sheath sensor 34 reports entry into the space 10, then an access instrument 36 (which was inserted with the sheath 32 or through a lumen 35 in the sheath 32 after the sheath 32 accessed cavity 10) is left in the cavity 10 while the insertion sheath 32 is removed. This embodiment is particularly useful in instances where, once access is confirmed, future confirmation is not required since the sensor is removed along with the sheath. This embodiment is most useful in instances where the instrument 36 will remain in place for a long period of time (e.g., an implantable device with long-term action) or where the desired profile of the instrument 36 is sufficiently small that inclusion of the anatomical space sensor into the instrument 36 itself becomes technically challenging and economically impractical.

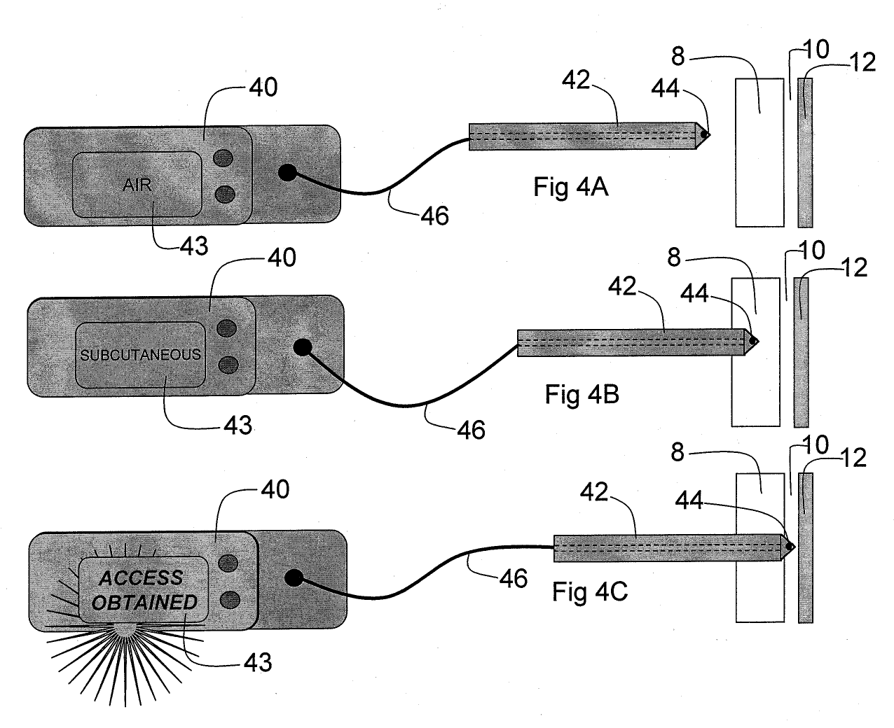

FIGS. 4A-C show an external reader 40 attached to an access device 42 via communication line 46. In this embodiment, the external reader 40 may have a display 43 or some other form of alert or indicator to let the user know that the access device has entered the correct cavity, or in which cavity the sensor currently resides. In its optimal embodiment, the anatomical space sensor 44 will provide information related to the tissue surrounding the sensor continuously and in real-time so that informed decisions to advance or retract the access device may be made. This illustration depicts the sensor incorporated within a removable insertion trocar, but it is important to note that this external reader and any other method of reporting device position to the user may be used with any of the sensing technologies described in herein.

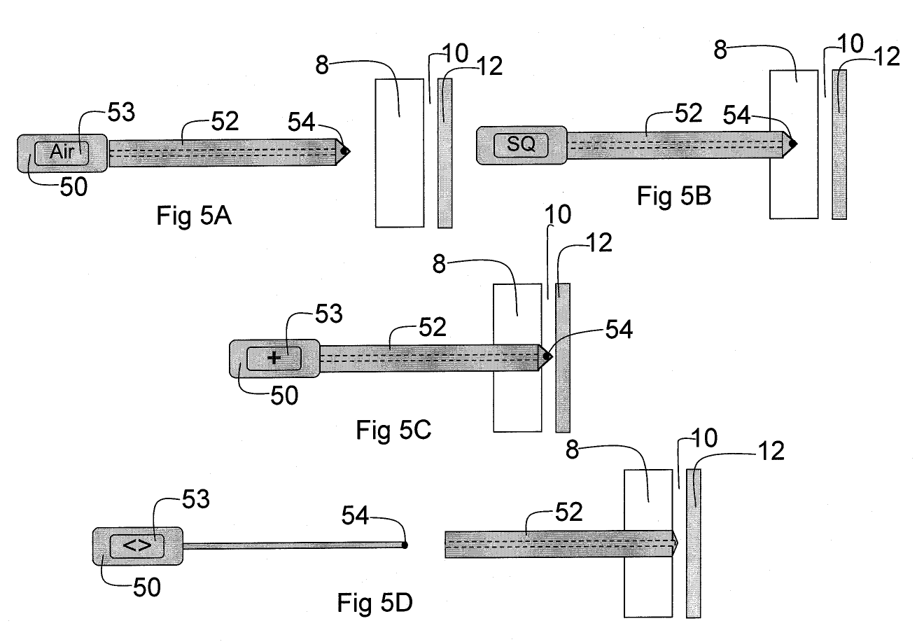

FIGS. 5A-D show an embodiment of an anatomical space access device in which a continuous reader 50 is incorporated into the access device, such as at a proximal end of the device's elongate body 52. In this embodiment, the integrated reader 50 may have a display 53 or some other form of alert or indicator to let the user know that the sensor 54 at the distal end of the access device has entered the correct cavity 10, or to identify the in cavity in which the sensor 54 currently resides. As with the external reader of FIG. 4, the sensor 54 will provide information related to the tissue surrounding the sensor continuously and in real-time so that informed decisions to advance or retract the access device may be made. This illustration depicts the sensor incorporated within a removable insertion trocar (as shown in FIG. 5D), but it is important to note that this external reader and any other method of reporting device position to the user may be used with any of the sensing technologies described herein. As with any of the embodiments described, the sensing device (here shown as the insertion trocar), may be disposable or reusable.

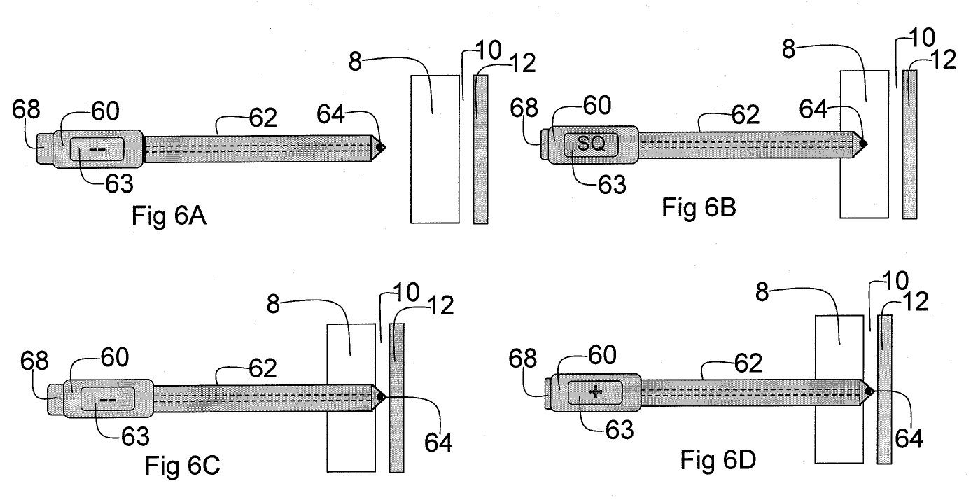

FIGS. 6A-D shows an embodiment in which an intermittent reader 60 is incorporated into the access device, such as at the proximal end of the device's elongate body 62. In this embodiment, the integrated reader 60 may have a display 63 or some other form of alert or indicator to let the user know that the sensor 64 at the distal end of the access device has entered the correct cavity 10, or to identify the cavity in which the sensor 64 currently resides. As with the integrated reader of FIG. 5, in one embodiment, the sensor 64 will provide information related to the tissue surrounding the sensor 64, but will do so only when activated, in this instance via deployment of a reversible push-button 68 at the proximal end of the insertion device, as shown in FIGS. 6B and 6D. This intermittent reading may give exact tissue location information and may be deployed repeatedly. This embodiment is particularly appealing for sensing technologies (e.g., optical technologies) that may produce heat or other potentially harmful byproducts and should only be activated for brief periods of time. As with other embodiments, informed decisions to the advancement or retraction of the access device may be made. This illustration depicts the sensor incorporated within a removable insertion trocar, but it is important to note that this external reader and any other method of reporting device position to the user may be used with any of the sensing technologies described herein. As with any of the embodiments described, the sensing device (here shown as the insertion trocar), may be disposable or reusable.

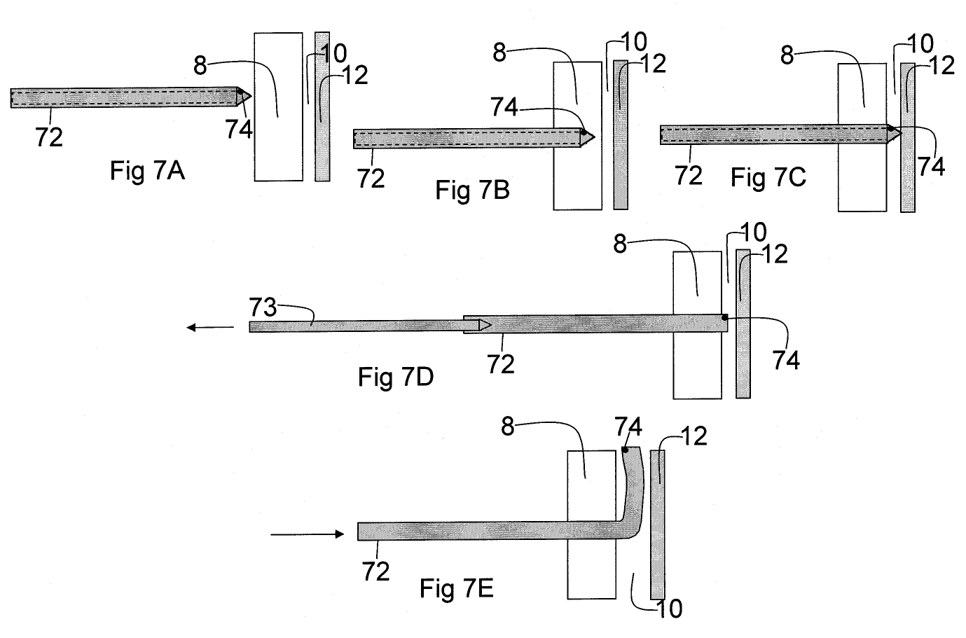

FIGS. 7A-E show an embodiment of the invention in which an anatomical space sensor 74 is incorporated into a catheter 72. A central trocar 73 (shown in FIG. 7D) may be used for initial placement of the catheter into the proper vessel or cavity 10. In contrast to the embodiment of FIG. 3, in this embodiment the insertion trocar 73 may be removed and the sensor-containing sheath or catheter 72 may remain within the cavity 10. This embodiment is particularly useful for catheter insertion and advancement, as shown in FIGS. 7A-E. Using the sensor 74 at the distal tip, catheter position may be continuously or intermittently assessed while it is advanced, thereby ensuring that the catheter is not only in position when the trocar is removed, but that it remains within the correct cavity while it is advanced. The catheter or sheath may be single or multiple lumen catheter and may employ a sensor incorporated into instrument or an external sensor. The catheter may also use additional sensors or lumens or other communication means to external sensors in order to provide the desire intervention or therapy.

One embodiment of this invention is a method of accessing the peritoneal cavity with a catheter as shown in FIG. 7. In this embodiment, the sensor-containing catheter may be advanced using a central trocar as a stiffening element. This insertion procedure may employ a blunt dissecting instrument or may utilize the Seldinger technique (or modification thereof). Once the catheter or sheath begins to move through the tissues, the sensor 74 at the distal tip may report position to the user, either intermittently or continuously, indicating which tissues are surrounding the sensor. Once the peritoneal cavity has been accessed, a reader (either external or integrated within the access device) reports that the cavity has been accessed via visual, auditory or tactile stimuli. The central trocar may then be removed and the catheter advanced, once again during continuous monitoring by the sensor in its optimal embodiment. If the catheter moves from the peritoneal cavity (e.g., into subcutaneous tissues, muscle, bowel or any other organ) or becomes surrounded by another fluid (blood, urine, etc.) then the sensor may report the change and indicate to the user that the device is no longer optimally placed and that further intervention (whether it be simply adjusting the catheter or performing further investigation) is required. Using this device and method, the user may ensure precise and consistent access to the peritoneal cavity not only upon insertion but for the duration of the placement of the device and through any required manipulations. This and other peritoneal catheters of this invention may also be weighted (e.g., at the tip) to ensure that the catheter sinks to the most dependent portion of the peritoneal cavity. By sinking to the most dependent portion of the peritoneal cavity, the catheter tip will have access to the large pool of fluid without obstruction by the fatty, floating omentum and mesentery.

While this description has focused largely on methods and devices for peritoneal insertion, this same procedure and method may be used to access any body cavity, tissue or space reliably and consistently. In using this technology, clinicians may be confident that their instrument resides in its desired space without the requirement for complex instrumentation or costly imaging techniques. For example, in one embodiment this method and device may be used in conjunction with any access device that currently requires imaging to confirm placement, but without the need for ionizing radiation. Examples of such devices include nasogastric tubes, central venous lines, chest tubes, feeding tubes, etc.

Communications between the sensor and display or instrument control unit may also be done wirelessly, e.g., via RFID or Bluetooth. In the instance where the catheter is a dual lumen catheter, one lumen may be used for fluid delivery while the other may be used for fluid return and a temperature and/or pressure sensor may be incorporated along its length, ideally closer to the fluid return tubing than the fluid delivery tubing.

Furthermore, the logic controller of the present invention may provide improved safety by monitoring for any of the deleterious changes expected with excess fluid flow into, e.g., the peritoneal cavity or vascular space. Examples of monitored parameters that may signal a warning or automatically result in an adjustment to rate of fluid infusion/extraction and/or fluid temperature include: electrocardiograph monitoring, electro-encephalograph monitoring, pulse oximetry (either internally or peripherally), peritoneal cavity compliance, intrathoracic pressure, intraperitoneal pressure, bladder pressure, rectal pressure, cardiac output, cardiac stroke volume, cardiac rate, blood flow (e.g., in superior mesenteric, celiac, renal or other arteries), pressure in veins (particularly those that empty into the IVC, e.g., the femoral vein), pressure in arteries (particularly those distal to the aorta, i.e. the femoral artery), blood oxygenation (e.g., in rectal mucosa, peripheral fingers and toes, etc.), whole body oxygen consumption, pH and arterial pO2 and any other parameter that shows a measurable change once the peritoneal or vascular spaces have been overloaded. These parameters, in particular, have been found to change with increases in peritoneal pressure with significantly negative impact on each parameter found at 40 mmHg, thus monitoring for these changes in conjunction with the peritoneal infusion catheter of the present invention will allow for even greater safety with peritoneal infusion. These parameters may be measured a variety of ways and the data transmitted either wirelessly or via wires to the logic controller in order to alert the healthcare provider or to automatically adjust the fluid flow/temperature in order to optimize both the flow of the peritoneal fluid and patient safety.

Exemplary methods of the invention include safe peritoneal access. The patient is prepared for paracentesis. An access system (such as one of those described above) is advanced through the subcutaneous and deeper tissues slowly while a reader indicates depth of insertion based, e.g., on a unique electrical signature of the tissue surrounding the anatomical space sensor (impedance, resistance, capacitance, etc.). Once the reader indicates that the peritoneal cavity has been accessed, advancement ceases, and a central insertion trocar may be removed. The soft, blunt-tipped catheter may then be advanced, if desired, while monitoring the reader. Once the catheter is at the desired location, an interventional procedure may be performed on the patient. If the catheter position is not correct, it may be repositioned. The anatomical space sensor may be monitored during the interventional procedure to ensure that the catheter tip has not migrated away from the desired location. The anatomical space sensor, or another sensor, may be used to indicate complications, such as the presence of blood. Other sensors may be used in addition to the anatomical space sensor, such as temperature or pressure sensors, to guide therapeutic intervention, such as optimization of peritoneal filling with peritoneal hypothermia or resuscitation.

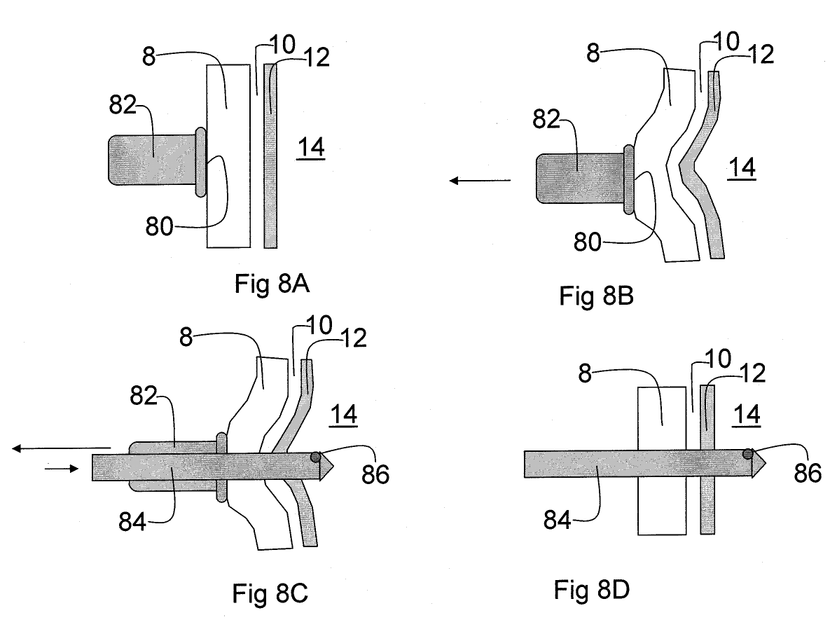

FIGS. 8A-D show a tenting mechanism for use with the anatomical space access device of this invention. This device may surround the site of access 80 (as shown), may be adjacent to the site of access or may engage multiple tissue sites around the access site. In one embodiment, the tenting mechanism has a circumferential handle 82 allowing a one-handed grip and for applying an abduction force while the cavity 10 is accessed by access device 84 (having anatomical space sensor 86) through the center of the tenting handle 82 (as shown in FIG. 8C), thereby providing optimal tissue elevation. The near circumferential design then allows the tenting mechanism to be removed from the insertion element or catheter 84 by an optional slot in its side (thus nearly circumferential). In this or any of the following embodiments, the tissue engagement portion of the tenting mechanism capable of providing an abducting force may consist of suction, adhesive application, epidermal puncturing elements or any other material or design that firmly (and, ideally, reversibly) captures tissue without damaging the underlying tissues. The tenting mechanism may also provide a rapid controlled skin incision at the center via a removable element (not shown) either upon deployment of a spring-loaded actuator or upon firm attachment of the tissue engagement element to, e.g., the abdominal wall to access a peritoneal cavity 14 through a peritoneal membrane 12. One embodiment of this design may involve the application of numerous micro-needles which provide minimal force, on their own, but in conjunction, provide enough force to easily lift the tissue.

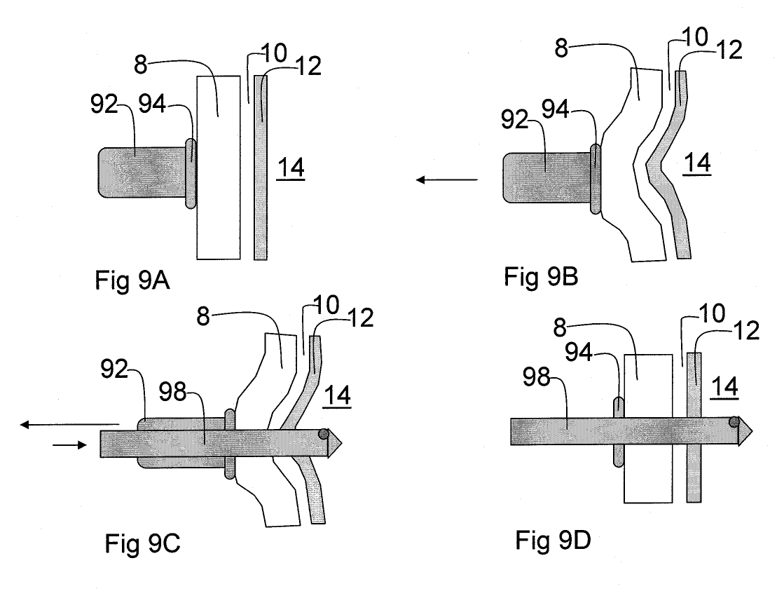

FIGS. 9A-D show another embodiment of a tenting mechanism for use with the anatomical space access device of this invention. In this embodiment, a tissue engagement portion 94 of the tenting mechanism may firmly engage the skin 8 prior to tenting, and the handle 92 may allow for firm abduction of the tissue. Once access has been obtained, the handle 92 may then be removed and the tissue engagement portion 94 left behind. FIGS. 9C and 9D show use of access device 98 (such as one of the devices described above) with the tenting mechanism.

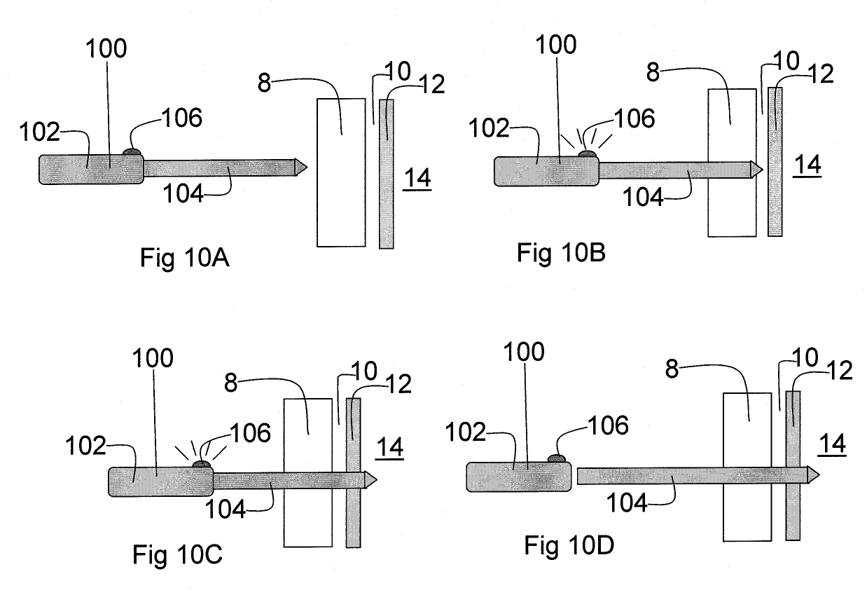

FIGS. 10A-D show an insertion force sensor 100 for use with an anatomical space access device of this invention. The insertion force sensor provides an indication to the user that the appropriate force is being applied during insertion. A force gauge 102 at the proximal end of the elongate body of the access device 104 (e.g., within a handle or within a trocar or needle) monitors the insertion force. An indicator 106 (such as a light) indicates either appropriate force or inappropriate force in alerting the user. As shown in FIG. 10D, the insertion force sensor 100 may be removed from the elongate body after insertion. This design is particularly important in conjunction with blunt trocars which may require twisting along with insertion driving force which is difficult for the user to judge due to the multiple planes of force. The alerting device may either be a part of the needle/trocar itself or may be removable.

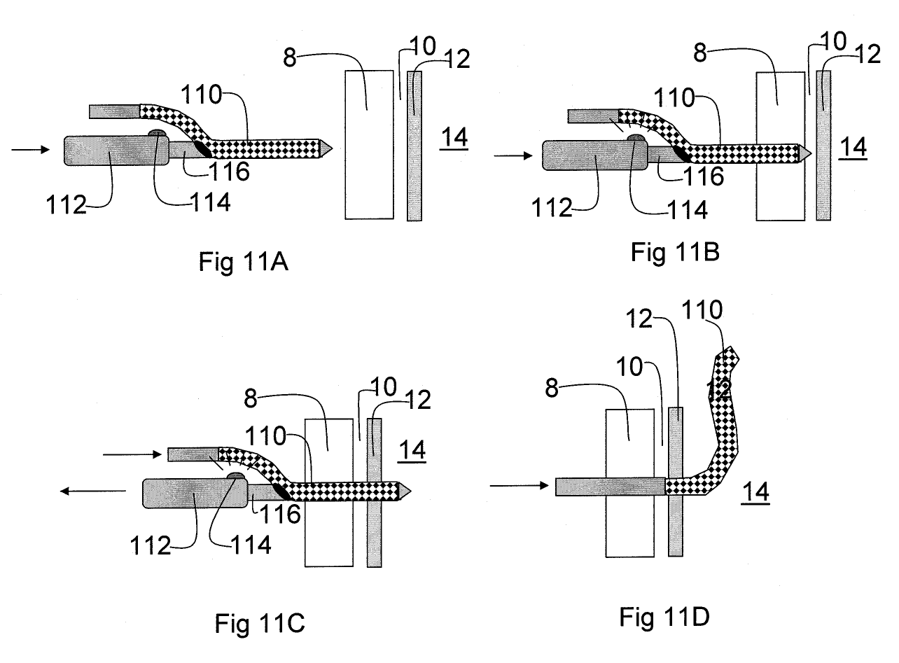

FIGS. 11A-D show another embodiment of an insertion force sensor 112 for use with an anatomical space access device, such as catheter 110. In this embodiment, a needle/trocar 116 is seen being used as a stiffening element for catheter insertion. The catheter 110 (here shown as a perforated drainage catheter) allows the trocar 116 through its lumen to puncture the tissue, or the trocar may run outside of the lumen. The catheter 110 may also have all or part of the blunt trocar tip incorporated into the catheter itself and reversibly engage the stiffening, force-sensing element 112. This element may also be capable of sensing cavity entry either via sensors on the catheter or the trocar/needle itself. Once access is obtained, the catheter may then be slid over the insertion element into the cavity to ensure protection of the underlying structures from the trocar itself.

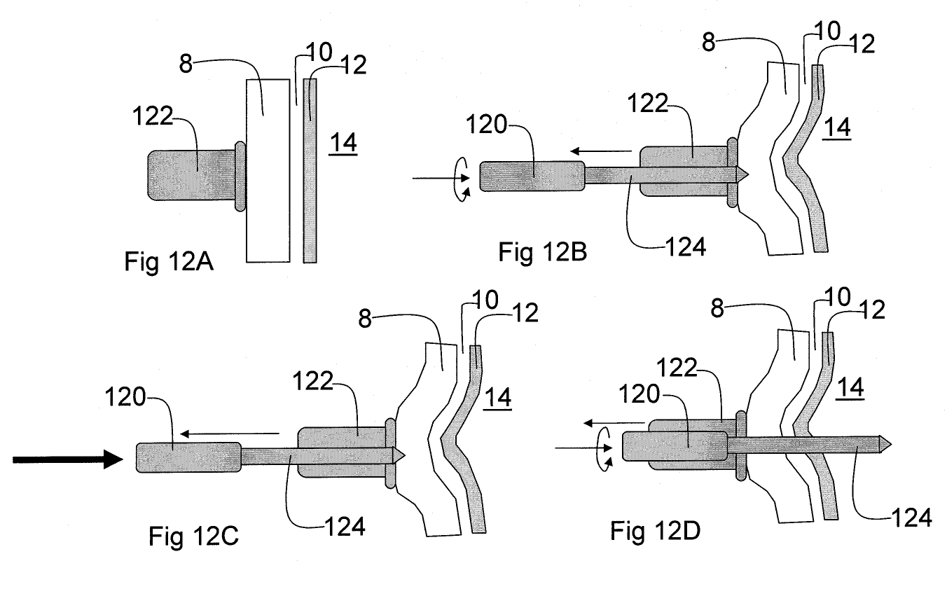

FIGS. 12A-D show a rotational feedback insertion sensor 120 being used with an optional tenting mechanism 122 and an anatomical space access device 124 according to this invention. In this embodiment, the insertion trocar or needle (ideally blunted and asymmetric) may be driven forward with any force, but may only be rotated along its axis (a critical component of the insertion process) with the appropriate application of driving force. Thus, the user will feel free slippage during rotation if excessive or inadequate force is applied and the rotational force will only be applied if the force is in the desired range. One embodiment of this design uses a force-sensitive spring which allows the shaft of the device to slide inside of the handle and, when appropriate force is applied, interdigitating element within the shaft to engage the handle and allow rotation. Other embodiments that similarly limit the force by preventing rotation with inappropriate force are also envisioned. In one embodiment this feature will be utilized in conjunction with the abdominal (or other cavity) tenting component of the device and may or may not include a cavity sensor to provide feedback that the cavity has been accessed to further prevent over-insertion. This rotational feedback mechanism and the force detector of FIG. 11 are particularly important for asymmetric trocar access. In this embodiment, rotational force is critical and is complicated by the requirement for additional driving force making training and implementation of there technologies difficult. The current invention allows technologies requiring rotational force for insertion to be inserted with an exquisite degree of control, including abdominal trocars/needles/catheters, bone marrow access devices, orthopedic instruments, venous or arterial access devices, or any other device requiring significant force for cavity entry. The trocar may be blunt, bladed, optical or blind. In yet another embodiment, an advancement control is provided under which one or both of either downward force on the elongate body or upward force with the tenting handle must be in the correct range for the elongate member to advance.

In the peritoneal cavity embodiment, the rotational element may, at its upper end, disengage and spin freely at a maximum of 5 mmHg insertion pressure, 10 mmHg insertion pressure, a maximum of 15 mmHg, a maximum of 20 mmHg, a maximum of 25 mmHg, a maximum of 30 mmHg, a maximum of 40 mmHg, a maximum of 50 mmHg, a maximum of 100 mmHg, a maximum of 200 mmHg, a maximum of 400 mmHg, and a maximum of 500 mmHg. For the 5 mm trocar embodiment, the rotational element may, at its upper end, disengage and spin freely at a maximum of 5 mmHg insertion pressure, 10 mmHg insertion pressure, a maximum of 15 mmHg, a maximum of 20 mmHg, a maximum of 25 mmHg, a maximum of 30 mmHg, a maximum of 40 mmHg and a maximum of 50 mmHg. In the peritoneal cavity embodiment, the rotational element may first engage the rotational element at a minimum of 5 mmHg insertion pressure, a minimum of 10 mmHg insertion pressure, a minimum of 15 mmHg, a minimum of 20 mmHg, a minimum of 25 mmHg, a minimum of 30 mmHg, a minimum of 40 mmHg, a minimum of 50 mmHg. For the 5 mm trocar embodiment, the rotational element may first engage the rotational element at a minimum of 5 mmHg insertion pressure, a minimum of 10 mmHg insertion pressure, a minimum of 15 mmHg, a minimum of 20 mmHg, a minimum of 25 mmHg, a minimum of 30 mmHg, a minimum of 40 mmHg, a minimum of 50 mmHg. In its optimal embodiment, the rotational element of the 5 mm trocar may first engage at an insertion pressure of 10 mmHg and then disengage at 30 mmHg to prevent over-insertion.

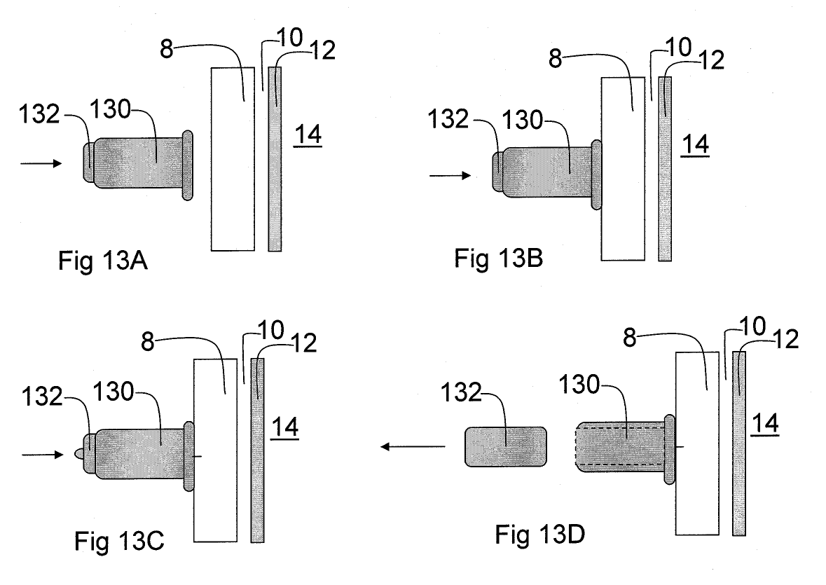

FIGS. 13A-D show an embodiment of a tenting mechanism with a removable incision element for use with the anatomical space access devices of this invention. In this embodiment, the central element 132 of the tenting mechanism 130 contains either a standard blade or a spring-actuated blade which can be deployed prior to removal of the central element. In this manner, the skin can be excised at the center of the tenting element once it has engaged the tissue to ensure that the blunt insertion trocar can easily pass through the incision in the epidermis and track through the subcutaneous tissues/muscle/etc. into the peritoneum. This mechanism also obviates the need for an open scalpel which can be a safety hazard and allows for a more controlled initial incision.

Methods of using a tenting mechanism with an anatomical space access device include the following. After the patient's skin is prepped, the tenting mechanism is applied to the puncture site. An asymmetric blunt-tipped trocar is rotated and advanced through the channel of the tenting mechanism as abduction force is applied to the skin by the tenting mechanism. Excessive or inadequate insertion or rotation force may be monitored with a force sensor and indicated to the user. Rotational force may be prevented if inadequate advancement force is applied. The trocar may have an anatomical space sensor, as described above. The trocar may also function as a removable stiffening element for a catheter, which may then be advanced over the trocar to reside in the cavity of interest. Once access has been obtained and the catheter advanced, the trocar, if no longer needed, may be removed, and large defects closed. The catheter may have an optional sensor to give an indication of proper placement within the cavity prior to or during the intervention, such as a peritoneal hyperthermia treatment.

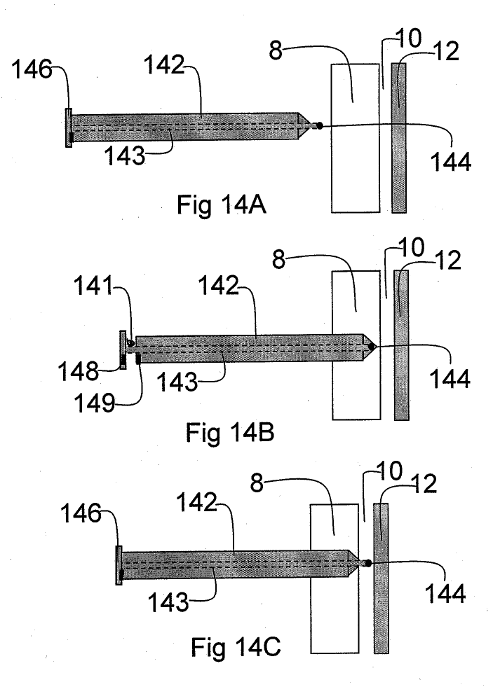

FIGS. 14A-C show yet another embodiment of an anatomical space access device. An elongate body 142 with an anatomical space sensor 144 at its distal end is shown entering a tissue volume 8. Sensor 144 is operably connected to a surface 146 at the proximal end of body 142 by a movable rod 143. A first contact 148 is disposed on surface 146, and a second contact 149 is disposed on the proximal end of elongate body 142. While outside of the tissue volume 8, as shown in FIG. 14A, sensor 144 extends distally from elongate body 142, and surface 146 is against the proximal end of elongate body 142. In this position, contacts 148 and 149 are in contact. As the distal end of the device enters tissue volume 8, the low tissue compliance of tissue volume 8 moves sensor 144, rod 143 and surface 146 proximally with respect to elongate body 142 against the action of a spring 141, thereby separating contacts 148 and 149. When the contacts are not in contact, an indication is provided to the user that sensor 144 is in tissue and not in a cavity. When sensor 144 passes into cavity 10 or other higher compliance tissue, however, spring 141 moves sensor 144, rod 143 and surface 146 distally forward, thereby bringing contacts 148 and 149 into contact and providing an indication to the user that the desired cavity has been reached.

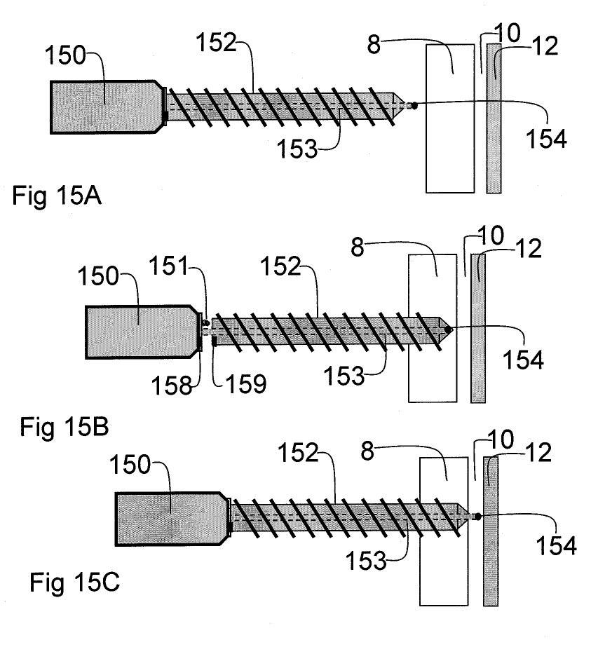

FIGS. 15A-C show an automated entry system in which an automated actuator 150 (such as, e.g., an electric rotary motor) turns a threaded trocar 152. As in the embodiment of FIG. 14, the elongate trocar body 152 with an anatomical space sensor 154 at its distal end is shown entering a tissue volume 8. Sensor 154 is operably connected to motor 150 at the proximal end of trocar 152 by a movable rod 153. A first contact 158 is disposed on motor 150, and a second contact 159 is disposed on the proximal end of trocar 152. While outside of the tissue volume 8, as shown in FIG. 15A, sensor 154 extends distally from trocar 152, and motor 150 is against the proximal end of trocar 152. In this position, contacts 158 and 159 are in contact. As the distal end of the device enters tissue volume 8, the low tissue compliance of tissue volume 8 moves sensor 154, rod 153 and motor 150 proximally with respect to trocar 152 against the action of a spring 151, thereby separating contacts 158 and 159. When the contacts are not in contact, an indication is provided to the user that sensor 154 is in tissue and not in a cavity. When sensor 154 passes into cavity 10 or other higher compliance tissue, however, spring 151 moves sensor 154, rod 153 and motor 150 distally forward, thereby bringing contacts 158 and 159 into contact and providing an indication to the user that the desired cavity has been reached. Alternatively, the forward motion of the penetrating member or trocar may be inhibited by inappropriately low or high levels of force on the penetrating member itself or on the tenting handle.

### EXAMPLE

Anatomical space access devices were constructed by mounting electrodes in the tip of a 5 mm plastic trocar. The electrodes were constructed by running wires through the trocar lumen, then soldering the wires at the tip to make an electrode. (In some prototypes, a first electrode was made by running a wire to the tip, then soldering it, and a second electrode was made by wrapping a wire around the shaft of the trocar tip.) Each electrode was then electrically connected to a capacitance meter. The meter was adjusted to the 200 microfarad range. A midline laparotomy was made in a recently sacrificed cadaveric pig, and a hand was inserted into the peritoneal cavity. A 1 cm incision was made in the animal's skin, then a blunt-tipped trocar was advanced while monitoring the capacitance at various levels and with varying force. The capacitance at the abdominal wall was measured, and the capacitance at the moment of entry into the peritoneal cavity (as verified by palpation using the hand in the peritoneal cavity) was recorded.

The average results of these measures (made in triplicate) were as follows:

| Level of insertion | Capacitance (picofarad) |
|---|---|
| Subcutaneous | 39.7 |
| Abdominal wall | 43.8 |
| Peritoneal membrane | 74.8 |
| Peritoneal cavity | 42.7 |

These data indicate the feasibility of detection of cavity entry by capacitance sensing with an average drop of over 40% in capacitance with cavity entry.

What is claimed is:

1. An anatomical space access device comprising:
an elongate body;
an insertion tip at a distal end of the elongate body;
an anatomical space sensor disposed at the distal end of the elongate body, the sensor being adapted to sense a parameter identifying an anatomical space other than a vasculature space and to generate a signal; and
an indicator operatively connected to the sensor to receive the signal and to indicate access of the sensor to the anatomical space.

2. The device of claim 1 wherein the insertion tip is blunt.

3. The device of claim 1 wherein the insertion tip is sharp.

4. The device of claim 1 wherein the sensor comprises an electrical property sensor.

5. The device of claim 1 wherein the sensor comprises a tissue compliance sensor.

6. The device of claim 1 wherein the sensor comprises a pressure sensor.

7. The device of claim 1 further comprising an insertion force sensor.

8. The device of claim 1 wherein the elongate body comprises a sheath, the device further comprising a trocar disposed within the sheath.

9. The device of claim 8 wherein the sheath comprises a weighted tip.

10. The device of claim 1 wherein the elongate body comprises a trocar, the device further comprising a sheath surrounding the trocar.

11. The device of claim 1 wherein the elongate body comprises a threaded trocar.

12. The device of claim 1 further comprising a tenting mechanism comprising a tissue attachment mechanism and a handle.

13. The device of claim 12 wherein the elongate body is disposed within the tenting mechanism.

14. The device of claim 13 wherein the elongate body is movably engaged with the tenting mechanism.

15. The device of claim 14 wherein the elongate body comprises threads and the tenting mechanism comprises threads engaged with the elongate body threads.

16. The device of claim 12 further comprising a tissue incision tool disposed within the tenting mechanism.

17. The device of claim 16 wherein the tissue incision tool comprises a spring biasing a sharp edge of the tool.

18. The device of claim 1 further comprising a rotation force sensor.

19. The device of claim 1 further comprising a rotation actuator engageable with the elongate body to rotate the elongate body and disengageable with the elongate body if a distally directed insertion force is below a threshold level.

20. The device of claim 1 wherein the anatomical space sensor comprises a distal tip mechanically connected to a first proximal contact such that the distal tip and first proximal contact are movable with respect to the elongate body, the anatomical space sensor further comprising a second proximal contact connected to a proximal end of the elongate body, the first and second proximal contacts having an open position in which the contacts are not in contact and a closed position in which the contacts are in contact.

21. The device of claim 20 wherein the anatomical space sensor further comprises a spring biasing the first and second proximal contacts toward the closed position.

22. The device of claim 20 further comprising an automated actuator operably connected to the elongate body to advance the elongate body only when the first and second proximal contacts are not in contact.

23. A method for providing access to an anatomical space outside of a vasculature space comprising:
inserting a distal end of an instrument through a tissue volume into the anatomical space outside of a vasculature space, the instrument comprising an anatomical space sensor; and
generating a location indication of the anatomical space sensor.

24. The method of claim 23 further comprising creating an opening in the tissue volume with the instrument.

25. The method of claim 24 wherein the instrument further comprises a blunt tip, the step of creating an opening comprises advancing the blunt tip through the tissue volume.

26. The method of claim 23 wherein the anatomical space is a peritoneal cavity.

27. The method of claim 23 wherein generating a location indication comprises sensing a parameter with the anatomical space sensor.

28. The method of claim 27 wherein sensing a parameter comprises sensing an electrical property.

29. The method of claim 27 wherein sensing a parameter comprises sensing temperature.

30. The method of claim 27 wherein sensing a parameter comprises sensing a change in tissue compliance.

31. The method of claim 27 wherein sensing a parameter comprises detecting a breathing pressure waveform.

32. The method of claim 23 further comprising sensing an insertion force during the inserting step.

33. The method of claim 32 further comprising indicating insertion force information.

34. The method of claim 23 wherein the instrument further comprises a blunt tip, the method further comprising pushing an anatomical structure aside during the inserting step.

35. The method of claim 34 wherein the anatomical structure is vasculature.

36. The method of claim 23 wherein the instrument comprises an insertion trocar and a sheath, the method further comprising removing the trocar and sensor after the inserting and generating steps.

37. The method of claim 23 wherein the instrument comprises a sheath, the method further comprising removing the sheath and sensor after the inserting and generating steps.

38. The method of claim 23 further comprising tenting the tissue volume prior to the inserting step.

39. The method of claim 38 wherein the tenting step comprises attaching a handle to the tissue volume.

40. The method of claim 39 wherein the inserting step comprises inserting the instrument through the handle.

41. The method of claim 38 further comprising preventing insertion of the instrument in the absence of a threshold tenting force.

42. The method of claim 23 wherein the inserting step comprises rotating the instrument and advancing the instrument distally.

43. The method of claim 42 wherein the rotating step comprises rotating a handle with a handle rotation force, the method further comprising disengaging transmission of the handle rotation force from the instrument if an advancement force is below a threshold level.

44. The method of claim 23 wherein the inserting step comprises inserting the instrument with an automated actuator, the method further comprising automatically ceasing operation of the automated actuator when the distal end of the instrument reaches a target cavity.
